(19) United States
(12) Patent Application Publication
(10) Pub. No.: US 2020/0030585 A1
(43) Pub. Date: Jan. 30, 2020

(54) Devices and Methods for Treating Congestive Heart Failure, Ascites, and Other Disorders Relating to Excess Bodily Fluid

(71) Applicant: Cook Medical Technologies LLC, Bloomington, IN (US)

(72) Inventors: Ghassan S. Kassab, La Jolla, CA (US); Zachary C. Berkwick, San Diego, CA (US); Matthew J. Phillips, San Diego, CA (US); Joshua Krieger, Topsfield, MA (US)

(73) Assignee: Cook Medical Technologies LLC, Bloomington, IN (US)

(21) Appl. No.: 16/338,450

(22) PCT Filed: Oct. 10, 2017
(86) PCT No.: PCT/US17/55859

§ 371 Date: Mar. 29, 2019

Related U.S. Application Data
(60) Provisional application No. 62/406,353, filed on Oct. 10, 2016.

(51) Int. Cl. A61M 27/00; A61M 39/02

(52) U.S. Cl. CPC A61M 27/002; A61M 39/0247; A61M 2039/0276; A61M 2039/0297; A61M 2039/027; A61M 2039/0291

(57) ABSTRACT

The present disclosure includes disclosure of devices, shunts, systems, and pump systems, as shown and/or described herein. The present disclosure includes disclosure of methods to treat conditions relating to excess bodily fluids and removal of the same, as referenced herein. A device or shunt can comprise a tube or catheter configured to facilitate drainage of fluid from within the abdomen into a portion of the excretory system. A device or shunt can include a cage/mesh at an external opening and can include a filter or membrane/barrier.

### BACKGROUND

The lymphatic system is known as the third circulatory system, having an extensive network of distensible channels that parallels the vascular systems and that drains into the veins. The lymph circulation collects and transports excess tissue fluid and extravasated plasma protein, absorbed lipids, and other large molecules from the intestinal space back to the venous system (jugular and subclavian veins) via the thoracic duct (TD). In particular, and under normophysiologic conditions, the thoracic duct drains into the left subclavian vein, and the right lymphatic duct drains into the right subclavian vein. However, under pathologic conditions, there may be an outflow obstruction or constriction. This may be anatomic or restrictive in regard to increased outflow resistance due to high lymphatic drainage in the presence of, for example, congestive heart failure (CHF) or other venous insufficiencies

In normal mammals, it is estimated that 40% of the total plasma protein pool and an equivalent fluid to the total plasma volume are returned to the blood through the TD each day at approximately 1 ml/min. Unlike the arterial and venous counterparts, the lymphatic system is much less characterized and hence provides enormous opportunities for discovery of novel diagnostics and therapeutics.

There are both diagnostic and therapeutic targets for TD interventions which were pioneered by Dr. Cope two decades ago (Cope, 1995; Cope et al, 1997). For the former, changes in flow pressure and composition of TD can aid differential diagnosis of various disorders such as metastatic cancer, intestinal tuberculosis, Whipple disease, hepatic cirrhosis, bacterial infections, parasites, fungi, etc. to name just a few. On the latter, there are three major classes of therapy via TD access: 1) Removal of excess fluid or decompression of lymphatic system, 2) Elimination of toxic substance dissolved in lymph, and 3) Depletion of cells circulating in the TD.

In view of the foregoing, the present disclosure includes disclosure to address the therapeutic targets, namely the decompression of the lymphatic system, so to treat CHF and other disorders relating to the lymphatic system.

### BRIEF SUMMARY

The present disclosure includes disclosure of a device or shunt configured to connect a portion of the lymphatic system to a portion of the circulatory system, the device or shunt configured to permit lymph from the lymphatic system to drain into the circulatory system.

The present disclosure includes disclosure of a device or shunt, comprising a one-way valve.

The present disclosure includes disclosure of a device or shunt, comprising a frame defining an entry opening and an exit opening.

The present disclosure includes disclosure of a device or shunt, comprising a filter or membrane/barrier.

The present disclosure includes disclosure of a device or shunt, used to treat a condition related to excess lymph within the lymphatic system and/or the inability or lessened ability to drain lymph from the lymphatic system into the circulatory system.

The present disclosure includes disclosure of a method to treat a condition related to excess lymph within the lymphatic system and/or the inability or lessened ability to drain lymph from the lymphatic system into the circulatory system, the method comprising the steps of delivering a device or shunt configured to connect a portion of the lymphatic system to a portion of the circulatory system into a mammalian body, the device or shunt configured to permit lymph from the lymphatic system to drain into the circulatory system; and positioning the device or shunt within the mammalian body so that the device or shunt connects the portion of the lymphatic system to the portion of the circulatory system so that lymph from the lymphatic system to drain into the circulatory system.

The present disclosure includes disclosure of a method to treat a condition related to excess lymph within the lymphatic system and/or the inability or lessened ability to drain lymph from the lymphatic system into the circulatory system, wherein the condition comprises congestive heart failure.

The present disclosure includes disclosure of a device or shunt configured to facilitate drainage of fluid from within the abdomen into a portion of the excretory system.

The present disclosure includes disclosure of a device or shunt, comprising a tube/catheter.

The present disclosure includes disclosure of a device or shunt, comprising a cage/mesh at an external opening of the device or shunt.

The present disclosure includes disclosure of a device or shunt, comprising a filter or membrane/barrier.

The present disclosure includes disclosure of a method to treat a condition related to excess fluid within the abdomen, the method comprising the steps of delivering a device or shunt configured to facilitate drainage of fluid from within the abdomen into a portion of the excretory system into a mammalian body; and positioning the device or shunt within the mammalian body so that the device or shunt permits fluid from the abdomen to drain into the excretory system.

The present disclosure includes disclosure of a method to treat a condition related to excess fluid within the abdomen, wherein the positioning step is performed to position the device or shunt into a wall of a ureter.

The present disclosure includes disclosure of a method to treat a condition related to excess fluid within the abdomen, wherein the positioning step is performed to position the device or shunt into a wall of a bladder and/or ureter.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a side view of a device having a one-way valve, according to an exemplary embodiment of the present disclosure;

FIG. 2 shows a device of the present disclosure positioned within the right lymphatic duct, according to an exemplary embodiment of the present disclosure;

FIG. 3 shows a device of the present disclosure positioned within the thoracic duct, according to an exemplary embodiment of the present disclosure;

FIG. 4 shows the cisterna chyli positioned relative to the left suprarenal vein, the left renal vein, the right suprarenal vein, and the right renal vein, according to an exemplary embodiment of the present disclosure;

FIG. 5 shows the cisterna chyli positioned relative to the azygos vein and the hemiazygos vein, according to an exemplary embodiment of the present disclosure;

FIGS. 6 and 7 show devices (such as a shunt) used to connect a portion of the lymphatic system to a portion of the circulatory system, according to exemplary embodiments of the present disclosure;

FIG. 8 shows devices (such as a shunt) positioned within a ureter and the bladder, according to an exemplary embodiment of the present disclosure;

FIG. 9 shows a device positioned within the bladder and a pump balloon used to direct fluid into the device, according to an exemplary embodiment of the present disclosure; and

FIG. 10 shows devices (such as a shunt) positioned within a stomach, according to an exemplary embodiment of the present disclosure.

An overview of the features, functions and/or configurations of the components depicted in the various figures will now be presented. It should be appreciated that not all of the features of the components of the figures are necessarily described. Some of these non-discussed features, such as various couplers, etc., as well as discussed features are inherent from the figures themselves. Other non-discussed features may be inherent in component geometry and/or configuration.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The present disclosure includes disclosure relating to the first therapeutic class (i.e., decompression of lymphatic system) with application to congestive heart failure (CHF) and other disorders.

The feasibility of TD lymph decompression/drainage has already been demonstrated in patients five decades ago (Dumont et al, 1963; Witte et al, 1969). Thoracic duct cannulation was made surgically in CHF patients (a total of 17 patients in two studies, mostly class IV stage) to allow drainage of the distended TD. The decompression therapy provided immediate resolution of a number of signs and symptoms, including significant reductions of the following: venous pressure, distention of veins, and peripheral edema. Ascites and hepatomegaly also diminished or resolved completely in those patients.

Despite the tremendous efficacy of this approach and relative safety, there are two major shortcomings, namely 1) required surgical access of TD, and 2) it only provides temporary relief as it does not address the root cause of lymphatic congestion. To reap a chronic therapeutic benefit, for example, the procedure must be repeated frequently. The first shortcoming has been addressed given the present non-surgical (percutaneous) access of the TD; however, a solution to the second shortcoming has previously not been addressed. The present disclosure addresses this second shortcoming, namely to provide a chronic therapeutic benefit previously unknown and unavailable in the medical arts.

To address this second shortcoming, a major question is what constitutes the bottleneck to drainage of lymphatic fluid into the venous system to avoid congestion/edema/ascites in CHF in the first place. This question can only be answered if we have an intimate understanding of the major determinants of lymphatic flow; namely: 1) resistance of lymphatic channels, and 2) the pressure gradient across the lymphatics. The former is dictated by the architecture (morphometry, branching pattern, etc.) and mechanical properties (passive compliance, active smooth muscle contraction, distribution of lymphatic valves, etc.) of the lymphatic system in health and in CHF. The latter requires an understanding of the hemodynamic conditions (pressure difference) between the lymphatic terminals and drainage veins.

Such an understanding has allowed us to design solutions for decompression of the lymphatic system as included in the present disclosure. Specifically, creation of thoracic duct-to-vein shunts can address the second shortcoming noted above. As drainage of the lymphatic system to the venous system as needed when a pressure gradient dictates the need. Such an implant, as referenced in further detail herein, can provide the chronic relief needed to maintain a decongested lymphatic system.

The basic premise is that an elevated systemic venous pressure in CHF reduces the pressure gradient for lymphatic flow and a connection to a lower pressure venous system can increase/restore the pressure gradient. The requirements of the device (diameter, lengths, opening/closing pressures, etc.), lymphatic and venous locations (e.g., TD-to-pulmonary vein given the lower pressure than the systemic veins where drainage normally occurs, Cole et al, 1967), etc., could only be determined rationally once the above noted characterization of the lymphatic system are made.

To address an anatomic outflow obstruction, the present disclosures includes disclosure of placement of either 1) a single stent, valve, or valve in stent (a “stent valve”), to open the restrictive thoracic duct while preventing (i.e. valve) backflow of venous blood into the lower duct, or 2) a series of valves placed individually or continuously within an extended bare metal or covered frame in order to promote lymphatic outflow by minimizing the upwardly pressure gradient similar to the function of native lymphatic valves. A stent, valve, or stent valve is referred to herein as a device 100, and a series/plurality of stents, valves, or stent valves are referred to herein as devices 100.

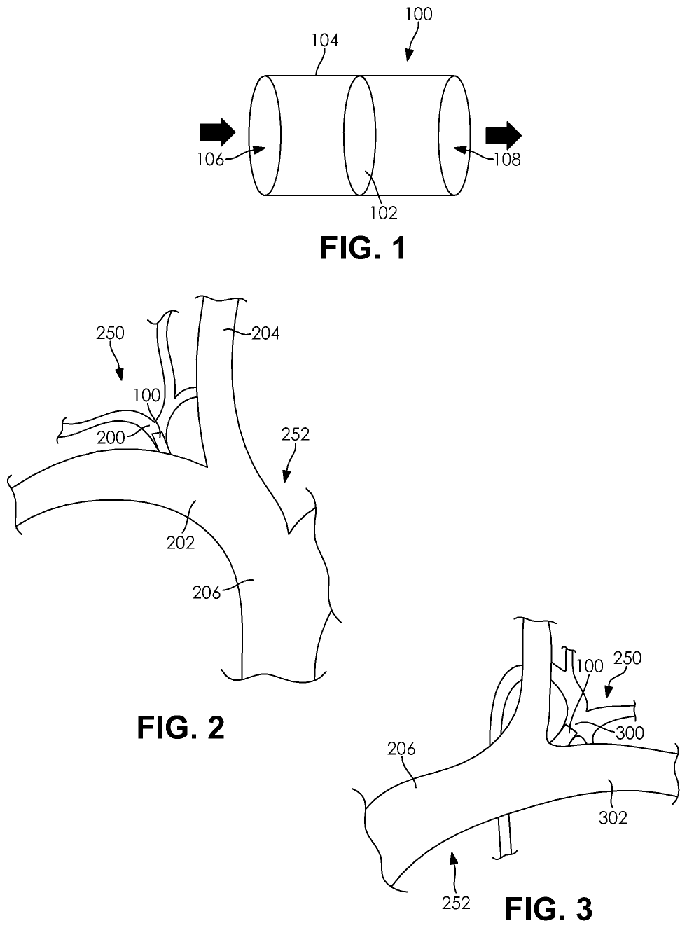

FIG. 1 shows an exemplary device 100 of the present disclosure. As shown therein, device 100 comprises a valve 102 configured to allow fluid flow therethrough in one direction, as indicated by the bold arrows in the figure. Valve 102, in various embodiments, is positioned within a frame 104 having/defining an entry opening 106 and an exit opening 108, whereby fluid, when valve 102 is positioned within the lymphatic system, can enter entry opening 106, pass through valve 102, and depart exit opening 108.

FIG. 2 shows an exemplary device 100 of the present disclosure positioned within the right lymphatic duct 200. With such a device 100 positioning, fluid from the right lymphatic duct 200 can flow into device 100, through valve 102 of device 100, and into the bloodstream, specifically into the right subclavian vein 202. The right internal jugular vein 204 and the brachiocephalic vein 206 are also shown in FIG. 2 for reference, noting that only portions of the lymphatic system 250 and the circulatory system 252 are shown in the figure. In view of the foregoing, an exemplary method of the present disclosure includes the step of positioning a device at least partially within the lymphatic system 250 of a mammal, whereby fluid from the lymphatic system 250 can drain into the circulatory system 252.

FIG. 3 shows an exemplary device 100 of the present disclosure positioned within the thoracic duct 300. With such a device 100 positioning, fluid from the thoracic duct 300 can flow into device 100, through valve 102 of device 100, and into the bloodstream, specifically into the left subclavian vein 302. The left internal jugular vein 304 and the brachiocephalic vein 206 are also shown in FIG. 3 for reference, noting that only portions of the lymphatic system 250 and the circulatory system 252 are shown in the figure. In view of the foregoing, an exemplary method of the present disclosure includes the step of positioning a device at least partially within the lymphatic system 250 of a mammal, whereby fluid from the lymphatic system 250 can drain into the circulatory system 252.

Given the configuration of device 100, valve 102 only permits fluid to drain from the lymphatic system 250, such as when used as noted above and as shown in FIGS. 2 and 3, into the circulatory system 252, but does not allow blood to backflow from the circulatory system 252 into the lymphatic system 250. Such a method allows lymphatic flow to occur from the lymphatic system 250 into the circulatory system 252 even in instances where blood pressure is relatively high, such as in CHF. As noted above, an elevated systemic venous pressure in CHF reduces the pressure gradient for lymphatic flow, and use of an exemplary device 100 of the present disclosure, as referenced herein, can increase or restore the pressure gradient necessary to allow fluid to drain from the lymphatic system 250.

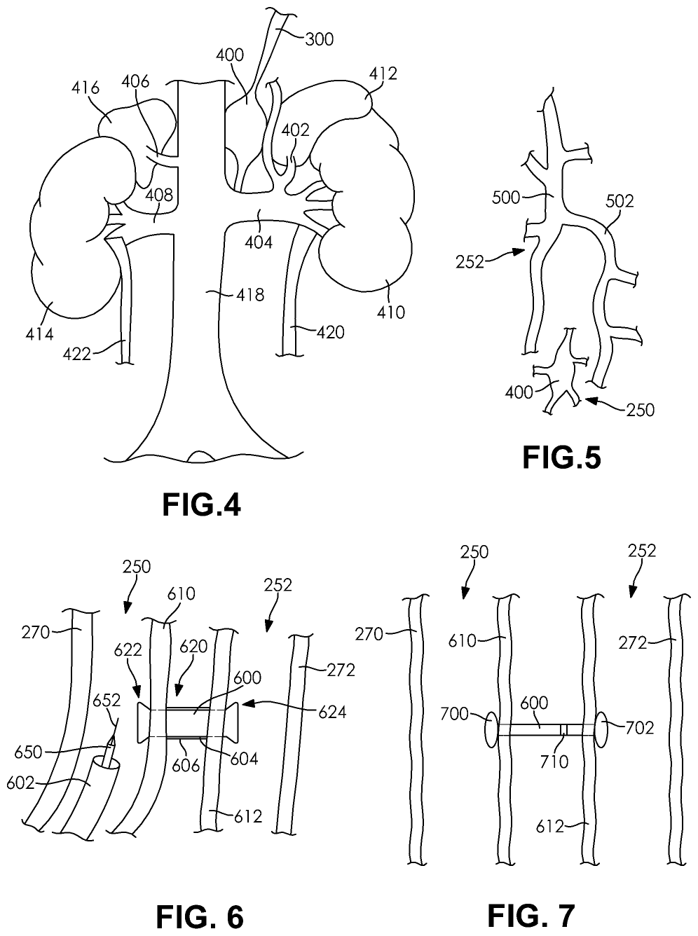

An alternative way of promoting outflow by decreasing resistance to antegrade lymphatic flow is to increase the cross sectional area of outlets. Specifically, in contrast to normophysiologic anatomy where the lymphatic system 250 drains solely into the left subclavian vein 302, additional locations for lymphatic-venous shunts may be created. A desired location for creating a shunt (in addition to the junction of the thoracic duct 300 and the left subclavian vein 302) can also include the cisterna chyli 400 to the left suprarenal vein 402, the left renal vein 404, the right suprarenal vein 406, and the right renal vein 408, such as shown in FIG. 4. FIG. 4 also shows the left kidney 410, the left adrenal gland 412, the right kidney 414, the right adrenal gland 416, and the inferior vena cava 418, for reference/context.

Another desired location for creating a shunt can include the cistema chyli 400 to the azygos vein 500 or the hemiazygos vein 502, such as shown in FIG. 5. Other portions of the lymphatic system 250 or the circulatory system 252 are not shown in FIG. 5.

For the applications referenced above and as shown in FIGS. 4 and 5, a shunt 600 (also referred to as a stent in various embodiments) is delivered over a catheter 602, such as shown in

FIG. 6, following establishment of transmural access across both vascular walls, namely a lymphatic vessel wall 610 and a circulatory vessel wall 612. A wire 652, a needle 650, or a needle 650 and a wire 652, can be used for the initial advancement within the circulatory system 250 to facilitate advancement of catheter 602 and ultimate delivery of shunt 600 (or device 100, as referenced herein). An exemplary shunt 600, in various embodiments, consists of a short frame 604 that may be covered with a coating 606, such as polyethylene terephthalate (PET or PETE) (such as DACRON®, for example), polytetrafluoroethylene (PTFE) (such as TEFLON®, for example), expanded polytetrafluoroethylene (ePTFE) (such as GORE-TEX®, for example), a biological material, etc., and forms a “dogbone” configuration once deployed to maintain apposition. As shown in FIG. 6, shunt 600 comprises a central portion 620, a first flared portion 622, and a second flared portion 624, whereby the first flared portion 622 is on an inside of a lymphatic vessel 270 of the lymphatic system 250 (through lymphatic vessel wall 610), and whereby the second flared portion 624 is on an inside of a circulatory vessel 272 of the circulatory system 252 (through circulatory vessel wall 612). The configurations (such as diameter and length) of the shunt 600 can be predetermined with SPY or CT imaging or intraoperatively with vascular imaging (fluoroscopy, intravascular ultrasound (IVUS), brightness (B) mode ultrasound, etc.). An exemplary device 100 or shunt 600 and at least one additional item, such as a catheter 602, a needle 650, and/or a wire 652, can be referred to herein as a system 680 or at least a portion of a system 680. Delivery can be made percutaneously, as generally referenced above, or laparoscopically, as may be desired for a given procedure.

In embodiments whereby shunt 600 comprises a one-way valve 102 such as shown in FIG. 1, shunts 600 can be referred to as exemplary devices 100 of the present disclosure. As such, an exemplary shunt 600 can comprises a frame 604 and a valve 102, in various embodiments.

In addition to methods described above, the lymphatic accumulation (namely the accumulation of lymph within the lymphatic system 250) may be shunted directly into the ureters and excreted in urine, or shunted directly into the stomach and excreted through the gastrointestinal system. This can be accomplished through a trans-venous lymphatic to ureter shunt or to a shunt directly into the stomach, as may be desired. Specifically, and regarding the ureters, either a lymphatic or uretal approach can be utilized. Similar to above, a wire 652, a needle 650, or a needle 650 and wire 652 can be used to puncture from the thoracic duct 300 (as previously referenced herein) into the renal veins (such as the left renal vein 404 or the right renal vein 408) and then into the ureter (such as the left ureter 420 or the right ureter 422), as shown in FIG. 4, or vice versa. With respect to a stomach shunt, a wire 652, a needle 650, or a needle 650 and wire 652 can be used to puncture from the thoracic duct 300 (as previously referenced herein) directly into the stomach 1000, as shown in FIG. 10. Either a covered stent or an in dwelling catheter (an exemplary device 100 or shunt 600) may be placed to establish flow. The flow can be made unidirectional through incorporation of a valve 102, such as in the form of a ball valve, bileaflet valve, or trileaflet valve, for example, either deployed after stent (device 100 or shunt 600) delivery or incorporated into the stent (device 100 or shunt 600) itself. In the case of an in dwelling catheter (shunt 600, shown in FIG. 7), a balloon may be inflated on either end to hold position, such as a first balloon 700 at one end of shunt 600 and a second balloon 702 at an opposite end of shunt 600, whereby first balloon 700 is inflated within lymphatic vessel 270 and whereby second balloon 702 is inflated within circulatory vessel 272. Balloon 702 may promote selective drainage if, for example, it occludes a branch or a portion of the lymphatic vessel to force unidirectional flow into, for example, a ureter 420, 422 for excretion. If anatomy permits, the device 100 or shunt 600 may be created directly from the thoracic duct 300 or the cisterna chyli 400 directly into a ureter 420, 422. Within either device (device 100 or shunt 600) a filter or membrane/barrier 710 may be used as part of device 100 or shunt 600 to limit/restrict cells (i.e. lymphocytes) or other undesired particulates from passing therethrough, namely to block the same from passing through device 100 or shunt 600 but not to retain/accumulate said particulates. In embodiments where filter or membrane/barrier 710 comprises a membrane or barrier, such a membrane or barrier would be configured with selective permeability so to, for example, allow plasma but not protein to pass therethrough, as in various embodiments it is desired that filter or membrane/barrier 710 does not capture any particulate matter.

The present disclosure includes the ability to seal/integrate with both the lymphatic system 250 and the circulatory system 252 boundaries/walls such that the perforations required to create the shunt do not communicate with the peritoneal cavity or other third spaces. This may be accomplished through compressive fit, mechanical interlock, adhesive, or tissue ingrowth/encapsulation, as may be desired.

In view of the foregoing, the present disclosure includes disclosure of devices 100 and shunts 600 useful to drain lymph from the lymphatic system 250 into the circulatory system 252 to treat a condition related to excess lymph within the lymphatic system 250 and/or the inability or lessened ability to drain lymph from the lymphatic system 250 into the circulatory system 252. CHF is one such condition. Said methods may be referred to herein as lymphatic decompression therapy.

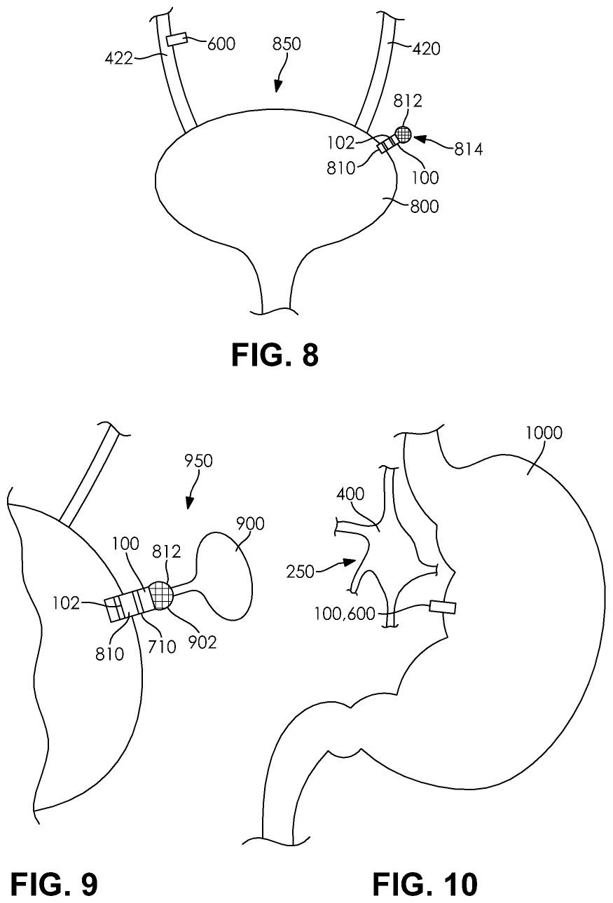

The present disclosure also includes disclosure of devices and methods for the removal of ascites, namely the abnormally accumulated fluid within the abdomen. Similar to approaches discussed above, a device 100 or shunt 600 may be created to drain ascites directly into the either the bladder 800 or a ureter 420, 422, such as shown in FIG. 8. This method involves placing a device 100 or shunt 600 having a one-way valve 102 into the wall of the bladder 800 or ureter 420, 422. Mechanisms for opening the valve 102 are pressure dependent and include, but are not limited to, the increase in abdominal pressure that occurs during inspiration or valvsalva maneuver. By use of pressure gradient alone, the valve 102 in device 100 or shunt 600 can be delivered transurethrally. Alternatively, opening of the valve 102 and flow of ascites across the short stented valve 102 (such as a ball valve, bileaflet valve, trileaflet valve, etc.) directed inwardly inside the bladder 800 can be achieved via an externally assisted device implanted laparoscopically. In at least one embodiment, device 100 comprises a tube/catheter 810 with a valve 102 therein, such as shown in FIG. 8. A cage/mesh 812, such as shown in FIG. 8, can form part of device 100, whereby cage/mesh 812 surrounds the external opening 814 of device 100 to prevent obstruction of omentum, etc. A secondary pump device can comprise a small tubular or flat balloon (referred to as a pump balloon 900, shown in FIG. 9) placed in between abdominal muscle layers such that during physical compression by the patient or abdominal contraction, fluid is expelled from opening 902 of balloon 900 and forced into the bladder 800, or ureter 420, 422, such as through a device 100 or shunt 600. A pump rate, for example, would be greater than the rate of generation, in that the rate of exhaust could be relatively slow, but would be relatively faster than the rate of fluid accumulation, and therefor sufficient to drain the entire space. For this approach, the valve 102 would be contained along the length of the tube/catheter 810 in an area that minimizes chances for obstruction, such as the lower ⅓ of tube/catheter 810 that extends from the balloon 900 to the bladder 800, as may be desired. A filter or membrane/barrier 710 can also be contained within tube/catheter 810 as may be desired. Pump balloon 902, such as shown in FIG. 9, is contiguous with device 100 or shunt 600 (meaning it is coupled/connected thereto). Pump balloon 900, for example, may be attached to device 100 or shunt 600 located in the ureter 420, 422 or bladder 800 as needed to facilitate pumping with appropriate placement of the pump balloon 900. A device 100 (or shunt 600) and a pump balloon 900 may be referred to collectively as a pump system 950 or at least a portion of a pump system 950.

In view of the foregoing, the present disclosure includes disclosure of devices 100 and shunts 600 useful to drain fluid from the abdominal cavity into the excretory system 850 to treat a condition related to excess fluid within the abdominal cavity and/or the inability or lessened ability to drain or otherwise get rid of excess fluid from the abdominal cavity. Ascites is one such condition.

Various devices 100 and shunts 600 of the present disclosure may comprise one or more features or elements as described herein with respect to the other. For example, FIG. 8 shows a device 100 having a cage/mesh 812, and a cage/mesh 812 can also be used with shunt 600. FIG. 1 shows a device 100 having a valve 102, and a valve 102 can also be used with a shunt 600, for example.

While various embodiments of devices and methods for treating congestive heart failure, ascites, and other disorders relating to excess bodily fluid have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

The present disclosure includes disclosure of devices, shunts, systems, and pump systems, as shown and/or described herein. The present disclosure includes disclosure of methods to treat conditions relating to excess bodily fluids and removal of the same, as referenced herein.

The present disclosure includes disclosure of a device or shunt configured to connect a portion of the lymphatic system to a portion of the circulatory system, the device or shunt configured to permit lymph from the lymphatic system to drain into the circulatory system.

A device or shunt of the present disclosure can comprise a one-way valve. A device or shunt of the present disclosure can comprise a frame defining an entry opening and an exit opening. A device or shunt of the present disclosure can comprise a filter or membrane/barrier.

A device or shunt of the present disclosure can be used to treat a condition related to excess lymph within the lymphatic system and/or the inability or lessened ability to drain lymph from the lymphatic system into the circulatory system.

The present disclosure includes disclosure of a method to treat a condition related to excess lymph within the lymphatic system and/or the inability or lessened ability to drain lymph from the lymphatic system into the circulatory system, the method comprising the steps of: delivering a device or shunt configured to connect a portion of the lymphatic system to a portion of the circulatory system into a mammalian body, the device or shunt configured to permit lymph from the lymphatic system to drain into the circulatory system; and positioning the device or shunt within the mammalian body so that the device or shunt connects the portion of the lymphatic system to the portion of the circulatory system so that lymph from the lymphatic system to drain into the circulatory system. The condition can comprise congestive heart failure.

The present disclosure includes disclosure of a device or shunt configured to facilitate drainage of fluid from within the abdomen into a portion of the excretory system. A device or shunt of the present disclosure, can comprise a tube/catheter. A device or shunt of the present disclosure can comprise a cage/mesh at an external opening of the device or shunt. A device or shunt of the present disclosure can comprise a filter or membrane/barrier.

The present disclosure includes disclosure of a method to treat a condition related to excess fluid within the abdomen, the method comprising the steps of: delivering a device or shunt configured to facilitate drainage of fluid from within the abdomen into a portion of the excretory system into a mammalian body; and positioning the device or shunt within the mammalian body so that the device or shunt permits fluid from the abdomen to drain into the excretory system. The positioning step of the method can be performed to position the device or shunt into a wall of a ureter. The positioning step of the method can be performed to position the device or shunt into a wall of a bladder and/or ureter.

### REFERENCES

Cope C. Percutaneous thoracic duct cannulation: feasibility study in swine. J Vasc Intery Radiol. 6(4):559-64, 1995.
Cope C., Timms I., Pavcnik D. Percutaneous transthoracic duct catheterization to the neck and esophagus: a feasibility study. J Vasc Intery Radiol. 8(5): 845-9, 1997.
Dumont, A. E., Clauss, R. H., Reed, G. E., Tice, D. A. Lymph Drainage in Patients with Congestive Heart Failure. Comparison with Findings in Hepatic Cirrhosis. N Engl J Med. 269: 949-52, 1963.
Witte M. H., Dumont A. E., Clauss R. H., Rader B., Levine N., Breed E. S. Lymph circulation in congestive heart failure: effect of external thoracic duct drainage. Circulation. 39(6):723-33, 1969.
Cole W. R., Witte M. H., Kash S. L., Rodger M., Bleisch W. R., Muelheims G. H. Thoracic duct-to-pulmonary vein shunt in the treatment of experimental right heart failure. Circulation. 36(4): 539-43, 1967.

We claim:

1. A device to facilitate drainage of fluid from within the abdomen of a mammal into a portion of the excretory system of said mammal, said device comprising:
a frame defining a lumen, an entry opening, and an exit opening;
a valve positioned within the lumen and configured to allow fluid flow therethrough from the entry opening to the exit opening;
a mesh surrounding the entry opening; and
a membrane barrier disposed within the lumen.

2. The device of claim 1, wherein the frame includes a first flared portion that defines the entry opening.

3. The device of claim 2, wherein the frame includes a second flared opening that defines the exit opening.

4. The device of claim 1, wherein the membrane barrier is disposed axially between the valve and the entry opening.

5. The device of claim 4, wherein the frame has a lower portion that comprises one third of the axial length of the frame and extends axially along the frame from the exit opening toward the entry opening; and
wherein the valve is disposed within the lower portion of the frame.

6. The device of claim 1, further comprising a pump balloon coupled to said device.

7. The device of claim 6, wherein the pump balloon is connected to the mesh.

8. The device of claim 7, wherein the pump balloon is configured to be positioned between abdominal muscle layers of said mammal such that during physical compression or abdominal contraction, fluid is expelled from the pump balloon and through the lumen of said frame.

9. The device of claim 1, further comprising a coating covering the frame.

10. The device of claim 9, wherein the coating is selected from the group consisting of polyethylene terephthalate, polytetrafluoroethylene, expanded polytetrafluoroethylene, and a biological material.

11. A device to facilitate drainage of fluid from within the abdomen of a mammal into a portion of the excretory system of said mammal, said device comprising:
a frame defining a lumen, an entry opening, and an exit opening;
a valve positioned within the lumen and configured to allow fluid flow therethrough from the entry opening to the exit opening;
a mesh surrounding the entry opening;
a membrane barrier disposed within the lumen axially between the valve and the entry opening; and
a pump balloon contiguous with said device and configured to force said fluid through the lumen from the entry opening to the exit opening.

12. The device of claim 11, wherein the frame includes a first flared portion that defines the entry opening.

13. The device of claim 12, wherein the frame includes a second flared opening that defines the exit opening.

14. The device of claim 11, wherein the frame has a lower portion that comprises one third of the axial length of the frame and extends axially along the frame from the exit opening toward the entry opening; and
wherein the valve is disposed within the lower portion of the frame.

15. The device of claim 11, wherein the pump balloon is connected to the mesh.

16. The device of claim 11, wherein the pump balloon is configured to be positioned between abdominal muscle layers of said mammal such that during physical compression or abdominal contraction, fluid is expelled from the pump balloon and through the lumen of said frame.

17. The device of claim 11, further comprising a coating covering the frame.

18. The device of claim 17, wherein the coating is selected from the group consisting of polyethylene terephthalate, polytetrafluoroethylene, expanded polytetrafluoroethylene, and a biological material.

19. A method to treat a condition related to excess fluid within the abdomen of a mammal, said method comprising the steps of:
delivering into a body of said mammal a device configured to facilitate drainage of fluid from within said abdomen into a portion of the excretory system of said mammal; and
positioning the device within the body of said mammal so that the device permits fluid from said abdomen to drain into the excretory system;
wherein the device comprises
a frame defining a lumen, an entry opening, and an exit opening;
a valve positioned within the lumen and configured to allow fluid flow therethrough from the entry opening to the exit opening;
a mesh surrounding the entry opening; and
a membrane barrier disposed within the lumen.

20. The method of claim 19, wherein the positioning step comprises positioning the device into a wall of a bladder or a ureter of the excretory system of said mammal.
